(19) United States
(12) Patent Application Publication
(10) Pub. No.: US 2020/0360028 A1
(43) Pub. Date: Nov. 19, 2020

(54) SURGICAL METHOD AND A BONE PUNCH INSTRUMENT FOR CREATING A BONE TUNNEL

(71) Applicant: Stefan EGGLI, Pierrafortscha (CH)

(72) Inventors: Janosch HÄBERLI, Bern (CH); Stefan EGGLI, Pierrafortscha (CH); Tom OVERES, Langendorf (CH)

(21) Appl. No.: 16/411,879

(22) Filed: May 14, 2019

(51) Int. Cl. A61B 17/16; A61F 2/08; A61B 17/17; A61B 90/00

(52) U.S. Cl. CPC A61B 17/1604; A61F 2/0811; A61B 2090/062; A61B 90/06; A61B 17/1764; A61B 17/1714

(57) ABSTRACT

The invention relates to a surgical method for creating a bone tunnel in target bone by utilizing a punch instrument which is sized and shaped for removal of a bone plug out of the target bone. By removing the bone plug, the femoral tunnel can be created. The punch instrument comprises an impaction handle and punch head having a bore. The punch head comprises a cutting edge arranged circumferentially around the bore at a cutting end of the punch head. By using of the punch instrument, a bone plug can be retrieved without creating bone drilling debris. Furthermore, the bone plug can be retrieved as a solid piece of graft, to be used to as a graft at another site in the body thereby promoting healing and reducing recovery time after surgery.

### TECHNICAL FIELD OF THE INVENTION

The present invention relates to a bone punch instrument for creating a bone tunnel in a target bone, such as the lateral or medial femoral condyle. The invention also relates to a bone punch instrument and to a bone punch instrument kit that can be used to create the bone tunnel.

### BACKGROUND OF THE INVENTION

In orthopedic interventions around the knee, bone plugs are often retrieved from a harvesting site and transferred to a target site. An example surgical intervention process for such a transplantation is the anterior cruciate ligament (ACL) reconstruction process, during which autologous tissues, such as tendons and ligaments, and bone blocks/plugs are used. One example technique for ACL reconstruction uses the quadriceps tendon and a patella bone plug in the reconstruction process. This arthroscopically performed surgery includes the following procedures.

Procedure 1: Quadriceps tendon graft harvesting

A central piece of the quadriceps tendon graft having a length of e.g. approximately 5 cm including an attached proximal patellar bone plug having a length of e.g. approximately 2 cm is harvested. This graft will serve as a replacement for the torn anterior cruciate ligament.

Procedure 2: Tibial tunnel creation

A tunnel is drilled from the anterior and proximal tibia toward the natural tibial foot-print of the ACL. Preferably a hollow drill is used in order to retrieve a bone plug, which can be placed back in the tibial tunnel, at a later step during the surgery. The tibial tunnel is shaped as a through bore having of a diameter of approximately 10 mm.

Procedure 3: Femoral tunnel preparation

A guide wire is placed through the femoral bone, defining the intended anatomical direction of the femoral tunnel. Using a cannulated drill, the guide wire is over-drilled and the tunnel is created. The femoral tunnel is shaped as a blind hole having a diameter of approximately 8 mm and a length of approximately 25 mm.

Procedure 4: Graft placement/ACL reconstruction

The patellar bone plug including the quadriceps tendon as retrieved in procedure 1 is impacted into the femoral tunnel. The quadriceps graft is pulled over the joint space, and fixated in the tibial tunnel. Fixation may be carried out using a screw for suture attachment or an interference screw. As a next step, the tibial tunnel is filled with the bone plug, obtained when creating the tibial tunnel.

As described, normally the femoral tunnel is created using a cannulated drill, guided by the femoral guide wire. By means of drilling, the bone is removed. The disadvantage of this technique is that drilling implicitly creates drilling debris. This bone debris is captured within the joint space and forms swimming bone sediments in the synovial joint fluid.

Several disadvantages are associated with these sediments/bone debris:
The joint needs to be washed thoroughly during the surgery, which prolongs surgery time.
Remaining residues need to be built down by the body.
The sediments drastically reduce the camera view during the arthroscopically performed surgery.
Good bone is lost, which could be used to fill the patella graft site, and promote healing.

### SUMMARY OF THE INVENTION

It is an object of the present invention to overcome at least some of the problems associated with bone tunnel creation techniques, which may include femoral tunnel creation techniques.

According to a first aspect of the invention, there is provided a method of creating a bone tunnel in a femoral condyle by using a bone punch instrument as recited in claim 1.

By using the punch instrument comprising an impaction handle and a punch head, a bone plug can be retrieved without creating bone drilling debris. Furthermore, the bone plug can be retrieved as a solid piece of graft, which may be used to augment another site in the body, and therefore to promote healing and reducing recovery time after surgery. The proposed method may me used e.g. for creating a bone tunnel in a lateral femoral condyle for anterior cruciate ligament reconstruction surgery or for creating a bone tunnel in a medial femoral condyle for posterior cruciate ligament reconstruction surgery.

According to a second aspect of the invention, there is provided a femoral bone punch instrument as recited in claim 12.

The proposed bone punch instrument has for instance the advantage that thanks to the modular structure of the bone punch instrument, the impaction handle may be used many times, and not necessarily with the punch head to create bone tunnels. The punch head may be changed every time when a bone tunnel is created. In other words, the punch head may be a single-use device, i.e. disposable, while the impaction handle may be a multiple-use device. In this manner, a cutting edge of the punch head is always sharp and it can be ensured that the punch head is sterile.

According to a third aspect of the invention, there is provided a bone punch instrument kit as recited in claim 21.

By changing the size of a punch head used in the process, the shape and/or size of the bone plug can be chosen as desired. Thus, the bone punch instrument may be used for target bones of various sizes.

Other aspects of the invention are recited in the dependent claims attached hereto.

### BRIEF DESCRIPTION OF THE DRAWINGS

Other features and advantages of the invention will become apparent from the following description of a non-limiting example embodiment, with reference to the appended drawings, in which:

FIG. 1 depicts a bone punch instrument in an exploded view;

FIGS. 2A and 2B depict a handle of the bone punch instrument of FIG. 1 in a perspective view and a cross-sectional view, respectively;

FIGS. 3A and 3B depict a punch head of the bone punch instrument of FIG. 1 in a perspective view and a cross-sectional view, respectively;

FIGS. 4A and 4B depict a centralization element of the bone punch instrument of FIG. 1 in a perspective view and a cross-sectional view, respectively;

FIGS. 5A to 5H depict the process of creating a femoral tunnel;

FIG. 6 depicts a flow chart summarizing the method steps of creating a femoral bone tunnel; and

FIGS. 7A-7D depict in perspective views a punch head holder for receiving the punch head for removing a bone plug out of the punch head during different phases of the bone plug removal process.

### DETAILED DESCRIPTION OF THE INVENTION

An embodiment of the present invention will now be described in detail with reference to the attached figures. This embodiment is described in the context of creating a hole in the femoral bone, and more specifically in the lateral femoral condyle, while at the same time allowing retrieving a bone graft element out of the created hole, but the teachings of the invention are not limited to this environment. The teachings of the present invention are equally applicable for creating holes in other bones as well. Identical or corresponding functional and structural elements which appear in the different drawings are assigned the same reference numerals.

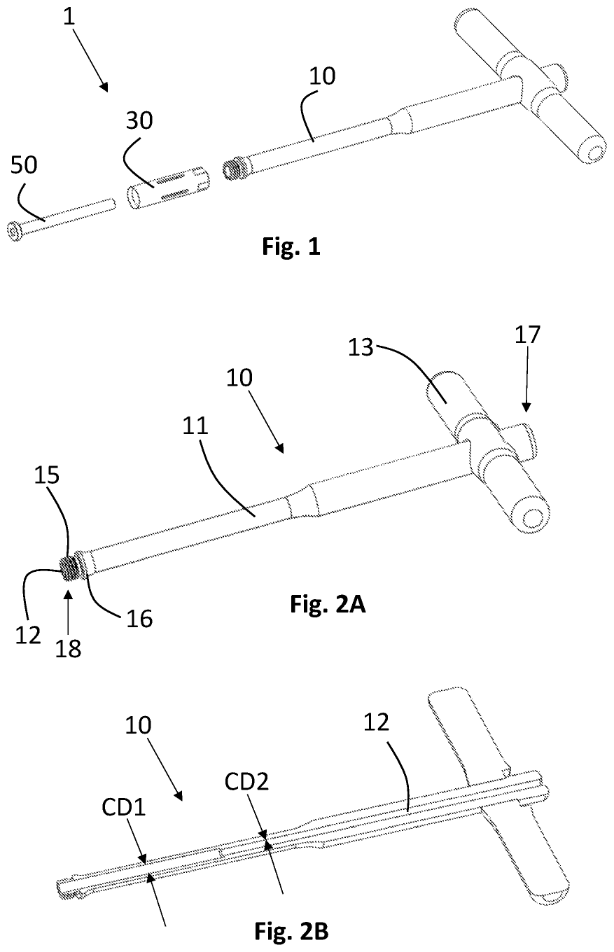

FIG. 1 shows the bone punch assembly, instrument or instrumentation 1 in an exploded view. In this example embodiment, the punching instrument comprises three physically separate elements, which may be connected or coupled to each other as explained later in more detail. As shown in FIG. 1, the interacting elements or instruments are an impaction handle or element 10, a punch head or element 30, and a centralization aid or element 50. It is to be noted that the centralization element is optional. Furthermore, according to one variant, the impaction handle and the punch head could be one single monobloc element, i.e. constructed from a single piece.

FIGS. 2A and 2B depict the impaction handle in a perspective view and a cross-sectional view, respectively. The impaction handle 10 comprises an elongated shaft 11 (defining a longitudinal axis, which may follow a straight or curved imaginary line) including a cannulation or channel 12 that extends through the shaft between a first end or extremity, referred to as a force receiving end or a proximal end 17, and a second end, referred to as a punch head coupling end or a distal end 18. In the present description, a proximal end refers to the end, which is closer to a user operating the instrument as it is intended to be used, whereas the distal end refers to the end away from the user. In the present embodiment, the impaction handle is a T-handle, comprising a grip element 13 which is oriented substantially perpendicular to the shaft. The grip element may in fact be manufactured as a physically separate element from the shaft so that the grip element, which is a straight or curved bar, is then firmly coupled (by e.g. welding, gluing and/or friction etc) to the shaft to form the impaction handle. In the present embodiment, the grip element has a through hole such that the shaft passes through the hole. Alternatively, the impaction handle can be a straight or bent element etc. A T-handle provides the advantage to comfortably be able to exert a torque on the target bone, to break off a bone plug as described in greater detail later.

The impaction handle comprises at its distal end 18 coupling means for coupling the impaction handle 10 to the punch head 30. The coupling means in this embodiment comprise a first connection or coupling element, and more specifically a first thread 15. However, a snap element or another friction connection element could be used instead. As can be seen e.g. in FIG. 2A, the first thread is in this example a male thread formed on the outer surface of the impaction handle 10. The first thread is shaped and sized to engage with a second thread 34 formed on the punch head 30. More specifically, the second thread is a female thread formed on the inner surface of the punch head at its proximal end 17. The distal end 18 of the impaction handle further comprises a seat 16 or a stop element sized and shaped to mate against the punch head 30 and more specifically the proximal end of the punch head 30. The seat forms an assembly limit and a force transfer area between the punch head and the impaction handle.

As shown in FIG. 2B, the cannulation 12 is a stepped cannulation, having more than one diameter. More specifically, the cannulation 12 comprises a first cannulation section having a first cannulation diameter CD1, and a second cannulation section having a second cannulation diameter CD2. In the present description when referring to a diameter of an object, the object does not necessarily have a circular cross section. The first cannulation section extends from the distal end toward the proximal end, while the second cannulation section extends from the proximal end toward the distal end. In this example, CD1 is greater than CD2. The first cannulation section is shaped and sized to receive in a substantially form fit manner and slidingly a portion of the centralization element as explained later in more detail. The second cannulation section is shaped and sized to receive in a substantially form fit manner and slidingly a guide wire as explained later in more detail. Alternatively, the cannulation 12 can have one single diameter or more than two cannulation diameters.

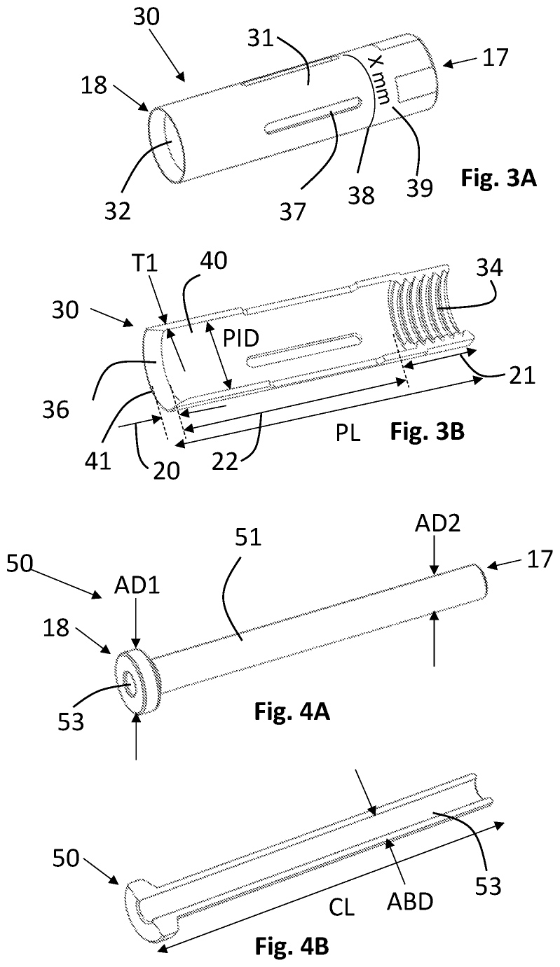

FIGS. 3A and 3B depict the punch head 30 (which may also be called a graft harvester) in a perspective view and a cross-sectional view, respectively. The punch head 30, which in this embodiment is a tubular and hollow element and has a substantially circular cross-section, comprises an elongated punch body 31 extending between a proximal end 17 (or an impaction handle coupling end) and a distal end 18, which also forms a punch cutting end. It is to be noted that the word tubular refers here to a tube-like shape, where the tube is a hollow and elongated body, but not necessarily having a circular cross section. A bore or channel 32 extends between the proximal end and the distal end. It is to be noted that in the present description, the word bore is understood to mean a hole, passage or tunnel irrespective of the manner the bore is created. For instance, the punch head could be made by three-dimensional printing without the need to create the bore by drilling. The proximal end 17 comprises in this embodiment a second coupling element, which is the second thread 34, which is shaped and sized to engage with the first thread 15 of the impaction handle 10. Thus, in this embodiment, the punch head is coupled with the impaction handle by means of a threaded connection. Instead of a threaded connection between the impaction handle and the punch head, other alternative coupling means may be envisaged, such as a snap connection or any other friction or form-fit connection. The punch head 30 has a punch head length PL. The punch head bore is delimited by a wall 40. At the distal end 18 of the punch head 30, there is provided a cutting edge 41 circumferentially around the bore and configured for bone cutting as explained later.

In this embodiment, the punch head 30 has a substantially cylindrical shape. The punch head can be divided longitudinally into three sections: a compression section 20 at the cutting end, a coupling element section 21 at the impaction handle coupling end, and a middle section 22 (or a thin-walled section) between the compression section and the coupling element section and having an inner diameter PID. The cross-sectional thickness of the middle section may be substantially constant. This thickness may be between 0.3 mm and 1.0 mm. The compression section a compression surface 36 formed by a punch head bore center facing surface of the wall. The compression surface is circumferentially tapered (and has thus a beveled end) toward a center of the punch head bore such that the compression surface is arranged to apply a compression force on the bone plug such that the compression force increases while the bone plug advances toward the impaction handle coupling end inside the punch head bore. This force is directed substantially toward a central (longitudinal) axis of the bone plug while the bone plug advances toward the proximal end of the bone plug. The compression force may thus be substantially orthogonal to the surface of the wall of the punch head. The length of the compression section along the longitudinal axis of the punch head may be between 0.5 mm and 20 mm or between 0.5 mm and 10 mm or between 1 mm and 5 mm or more specifically between 2 mm and 5 mm. The wall thickness and the cutting edge largely define the bone penetration resistance. As shown in FIG. 3B, in the compression section, the wall 35 is tapered toward the inner part of the punch head (its center) such that the cross-sectional thickness of the wall in the compression section gradually increases toward the proximal end of the punch head 30 (or the cross-sectional thickness of the wall gradually decreases toward the cutting edge 41) until it reaches a substantially constant thickness in the middle section. The punch head 30 shaped in this manner thus provides inwardly directed compression of the bone plug. However, it is to be noted that the compression surface could be designed so that the cutting edge is substantially flush with the inner wall of the middle section, and the compression surface would then taper toward the outside.

As is shown in FIG. 3A, the punch head may also comprise indications to identify the punching depth, such as openings, slots or windows 37, lines 38, text (i.e. numbers and/or letters) 39. The openings also allow increasing the friction between the punch head and the bone plug so that the punch head is not allowed to rotate around the bone plug to allow it be broken off as explained later in more detail. The punch head may also include one or more ribs, such as longitudinal ribs, which may extend longitudinally along the inner wall of the punch head. Instead, or in addition, other protrusions or bumps may be provided on the inner wall. The purpose of these ribs or protrusions is also to increase friction between the bone plug and the punch head.

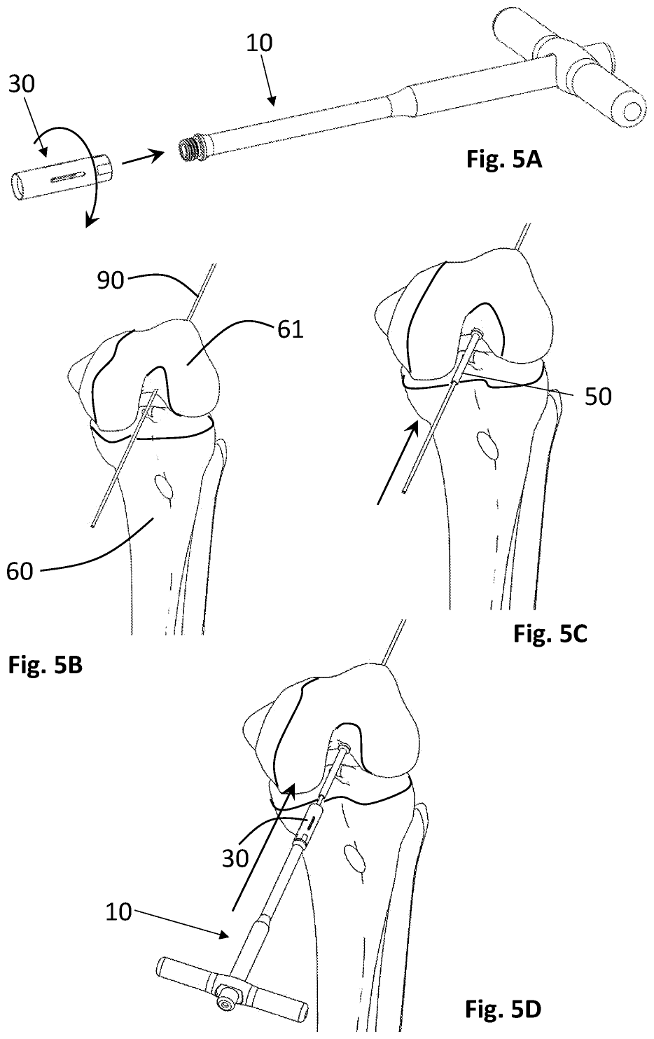

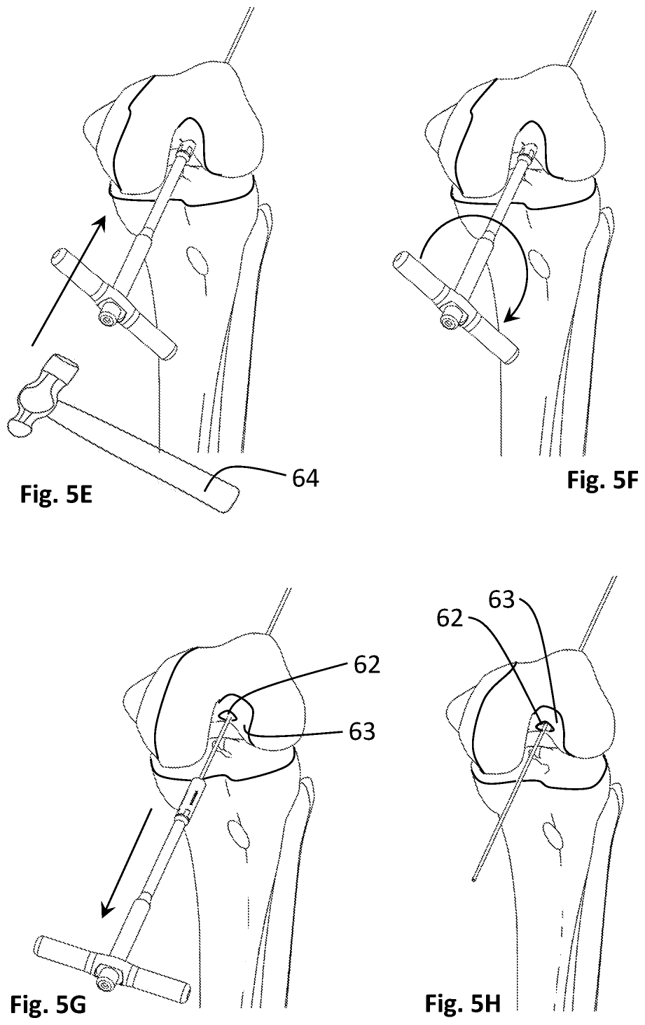

FIGS. 4A and 4B depict the centralization element 50 in a perspective view and a cross-sectional view, respectively. The centralization element 50 comprises an elongated centralization element body 51 extending between the proximal end 17 and the distal end 18 or a bone engaging end. As shown in FIGS. 4A and 4B, the elongated centralization element body 51 is a stepped body, having more than one external diameter. More specifically, the centralization element can be longitudinally divided into a first centralization element section characterized by a first centralization element outer diameter AD1, and a second centralization element section characterized by a second centralization element outer diameter AD2, which in this example is smaller than the first centralization element diameter. The first centralization element section is sized and shaped to be slidingly and in a form-fit manner received within the punch head bore and more specifically its middle section, and the second centralization element section is sized and shaped to be at least partially slidingly and in a form-fit manner received within the cannulation 12 of the impaction handle and more specifically in its first cannulation section. Alternatively, the centralization element body has one single external diameter. The centralization element 50 has a length CL. In this embodiment the length CL is greater than the length PL. Moreover, the centralization element 50 comprises a bore or channel 53 extending between the proximal end 17 and the distal end 18 and thus the centralization element bore extends completely through the elongated centralization element body 51. The bore 53 has at least one bore diameter ABD. In the present embodiment, the bore 53 is sized and shaped to receive a guide wire 90 (shown in FIGS. 5A to 5H) in a substantially play free manner. Thus, the diameter ABD may substantially equal to the diameter CD2. Alternatively, the bore 53 may be shaped as a non-cylindrical channel. For manufacturing cost reasons, it is advantageous that the centralization element 50 is shaped rotationally symmetrical. Alternatively, the cross section of the centralization element may be of any other shape, such as a quadratic shape. The centralization element 50 is in the present embodiment designed so that it can be entirely received a channel formed by the punch head bore 32 and the first cannulation section. Thus, a portion of the centralization element may also be received in the cannulation 12. As the periphery of the thickened portion of the centralization element is in this case designed to substantially mate with middle section of the punch head bore 32 or with its inner wall 40, the bore 53 is centrally aligned in the punch head bore 32.

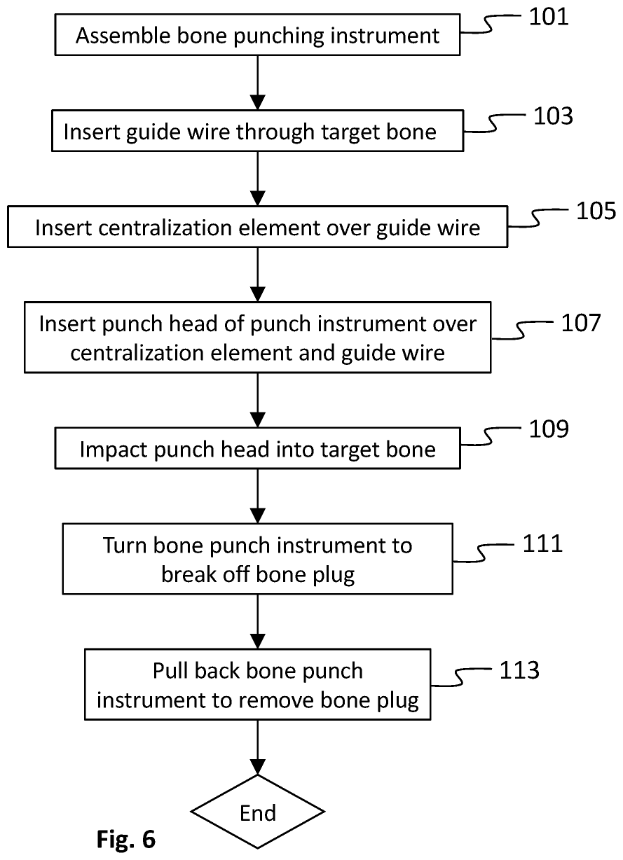

Referring to FIGS. 5A-5H and to the flow chart of FIG. 6, the femoral tunnel creation and the graft retrieval surgical steps are explained. For illustration purposes, tendons, ligaments, and the patella bone are blended out, providing a clear view of the tibial bone 60 and the femoral bone 61. It is to be noted that the method as described below may also be applied to other bones instead. In present embodiment, the below process may be used as a part of an ACL reconstruction process.

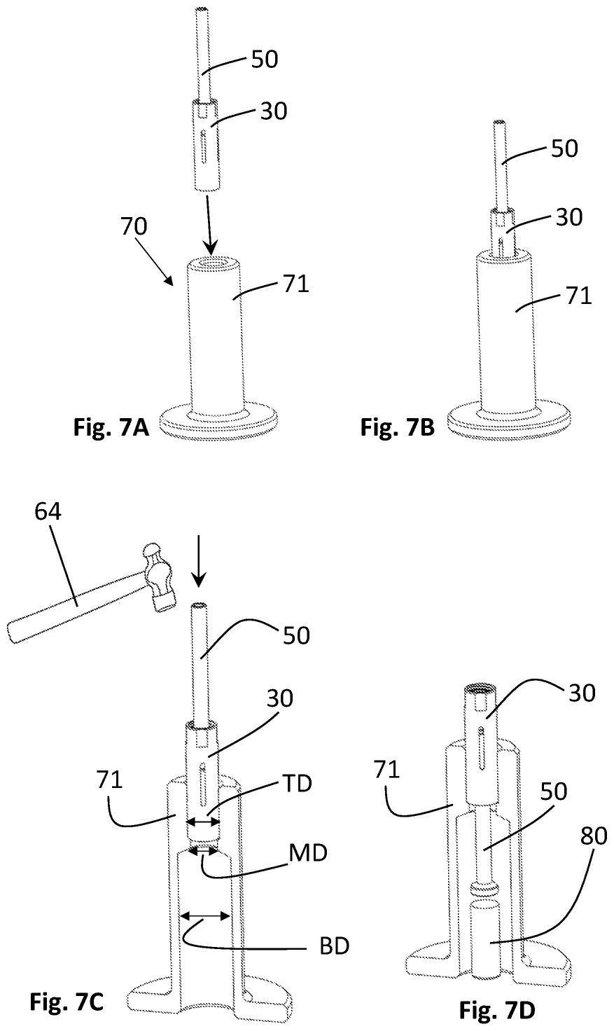

In step 101 and as shown in FIG. 5A, the bone punch instrument 1 is assembled. More specifically, in this step the punch head 30 is connected to the impaction handle 10. In this specific example, the impaction handle is screwed into the punch head so that they are firmly coupled together. In step 103 and as shown in FIG. 5B, a guide wire 90 is placed by drilling through the femoral bone 61 also referred to as a target bone. In step 105 and as shown in FIG. 5C, the centralization element 50 is placed over the guide wire 90, and it is advanced forward until it comes in contact with the femoral bone 61. It is to be noted that this step is optional. In step 107 and as shown in FIG. 5D, the punch head 30 when coupled with the impaction handle 10 is advanced forward over the guide wire 90 and the centralization element 50. The arrow in FIG. 5D shows the direction of advancement. In step 109 and as shown in FIG. 5E, the punch head 30 is forced into the target bone 61 and in this example into the inner side of the lateral femoral condyle also known as the lateral sidewall of the intercondylar notch, which may be considered to be part of the lateral femoral condyle or adjacent to the lateral femoral condyle. For this purpose there is provided a hammer or mallet 64, which is used by the operating person to hit or tap the proximal end of the impaction handle 10 to exert a force on the punch head. The direction of the force, which is toward the target bone, is shown in FIG. 5E by a solid arrow and it substantially coincides with the longitudinal axis of the guide wire. The coupling between the impaction handle and the punch head is such that the force exerted on the force receiving end of the impaction handle is configured to be transferred onto the punch head. The guide wire 90 and the centralization element 50 facilitate guidance, and therefore all elements or instruments are centrally aligned with respect to each other. In this example, the punch head 30 is predominantly received in the target bone once impacted to the correct depth so that a small portion of the punch head still protrudes out of the target bone. In step 111 and as shown in FIG. 5F, the operating person rotates the impaction handle. This rotation also causes the punch head 30 to turn in the same direction to break off the bone plug 80 (shown in FIG. 7D). The impaction handle is turned clockwise (when seen from the operator) if a counterclockwise rotation is required to decouple the impaction handle from the punch head to break off the bone plug, and the impaction handle is turned counterclockwise if a clockwise rotation is required to decouple the impaction handle from the punch head to break off the bone plug. The turning thus takes places about the longitudinal axis of the shaft of the impaction handle and/or the punch head. Once the bone plug has been broken off, the bone plug remains in the punch head bore 32. In step 113 and as shown in FIG. 5G, the bone plug 80 is removed out of the target bone by pulling back the impaction handle 10. As the punch head 30 is coupled with the impaction handle 10, also the punch head 30, the centralization element 50, and the bone plug 80 are pulled back together with the impaction handle 10. FIG. 5H shows the created femoral tunnel 62, which in this case is a blind hole, at the inner side of the femoral condyle 63. It is to be noted that in the above process, step 101 may be carried out at any moment before carrying out step 107. The created bone tunnel typically has a length of 15 mm-35 mm while the largest cross-sectional diameter is between 7 mm and 11 mm. It is to be noted that the punch head does not need to have a circular cross-section.

Referring to FIGS. 7A to 7D, a support instrument or punch head holder 70 is shown, which may be used to remove the bone plug from the punch head 30. The punch head holder may be considered to be part of the bone punch instrument or it can form an assembly or a system together with the bone punch instrument. However, it is to be noted that the punch head holder is an optional element that can be used to facilitate the removal of the bone plug out of the punch head but other suitable ways may be used instead for the bone plug removal. In this example, the punch head holder 70 has a tower-like body 71, and comprises a central stepped pocket or bore 72 at least partly extending longitudinally through the punch head holder. In this example, the pocket is a through bore inside the punch head holder. Furthermore, in this example, the stepped pocket has a cylindrical cross section, and has three diameters (although two or more diameters would equally be possible), namely a top diameter TD, a middle diameter MD and a bottom diameter BD. In this example, The top diameter TD is greater than the middle diameter MD, while the top diameter is smaller than bottom diameter BD. As shown in FIGS. 7A and 7B, the punch head 30 including the bone plug 80 and the centralization element 50 is placed in the punch head holder so that the punch head 30 is at least partially received in the pocket and more specifically in the upper pocket section having the top diameter. The middle diameter MD of the middle section of the punch head holder is smaller than the outer diameter of the punch head. In this manner, the middle section forms a stop for the punch head 30 and thus the punch head when inserted into the pocket from the top cannot advance beyond this stop. In fact, when in the pocket, the punch head 30 rests against the stop. The centralization element 50 extends out of the punch head 30 and more specifically out of its proximal end, and thus the centralization element forms a removal plunger for the bone plug 80. By means of for example tapping on the centralization element 50 and more specifically on its proximal end, the bone plug 80 is pressed out of the punch head 30, and captured in the punch head holder 70. The tapping may be executed by using the mallet 64. The bone plug may now be retrieved, and for example placed in the patella grafting site (not shown).

The invention also proposes a femoral bone punch instrument set (or kit) comprising the femoral bone punch instrument as described above and further comprising instructions for using the set. The instructions may thus comprise instructions for carrying out one or more of the steps explained in the flow chart of FIG. 6.

While the invention has been illustrated and described in detail in the drawings and foregoing description, such illustration and description are to be considered illustrative or exemplary and not restrictive, the invention being not limited to the disclosed embodiment. Other embodiments and variants are understood, and can be achieved by those skilled in the art when carrying out the claimed invention, based on a study of the drawings, the disclosure and the appended claims.

In the claims, the word “comprising” does not exclude other elements or steps, and the indefinite article “a” or “an” does not exclude a plurality. The mere fact that different features are recited in mutually different dependent claims does not indicate that a combination of these features cannot be advantageously used.

1. A method of creating a bone tunnel in a femoral condyle for anterior or posterior cruciate ligament reconstruction surgery by using a bone punch instrument comprising an impaction handle and a hollow, elongated punch head comprising a punch head bore and a cutting edge arranged circumferentially around the bore at a cutting end of the punch head, the method comprising
exerting a force on the impaction handle toward the femoral condyle to thereby cause the cutting edge to cut into the femoral condyle causing the punch head to be at least partially advanced into the femoral condyle;
turning the impaction handle to thereby cause the punch head to turn together with the impaction handle so that a portion of the femoral condyle, referred to as a bone plug, received in the punch head bore breaks off; and
pulling back the impaction handle to thereby remove the punch head and the bone plug out of the created bone tunnel.

2. The method according to claim 1, wherein the bone tunnel is created in a lateral femoral condyle or in a medial femoral condyle starting at a sidewall of an intercondylar notch.

3. The method according to claim 1, wherein the punch head is advanced into the femoral condyle by using a mallet or hammer.

4. The method according to claim 1, wherein prior to advancing the punch head into the femoral condyle, a guide wire is placed in or through the femoral condyle defining a direction of the bone tunnel.

5. The method according to claim 4, wherein prior to advancing the punch head into the femoral condyle, a centralization element is placed around the guide such that the centralization element is at least partly received in the punch head bore for centralizing the punch head around the guide wire.

6. The method according to claim 5, wherein after pulling back the impaction handle, the bone plug is removed out of the punch head by moving the centralization element toward the cutting end.

7. The method according to claim 1, wherein prior to advancing the punch head into the femoral condyle, the punch head is coupled to the impaction handle so that the force exerted on the impaction handle is configured to be transferred onto the punch head.

8. The method according to claim 1, wherein the created bone tunnel has a length of 15 mm-35 mm.

9. The method according to claim 1, wherein the created bone tunnel has a largest cross-sectional diameter of 7 mm-11 mm.

10. The method according to claim 1, wherein the created bone tunnel has a substantially circular cross-section.

11. The method according to claim 1, wherein the created bone is a blind hole.

12. A femoral bone punch instrument for creating a bone tunnel in a femoral condyle, the bone punch instrument comprising
an impaction handle having a force receiving end and a punch head coupling end comprising a first coupling element;
a hollow, elongated punch head having a cutting end and an impaction handle coupling end comprising a second coupling element configured to be removably coupled to the first coupling element such that a force exerted on the force receiving end of the impaction handle is configured to be transferred onto the punch head, the punch head comprising a punch head bore for receiving a bone plug of the femoral condyle, and a cutting edge arranged circumferentially around the bore at the cutting end of the punch head, the punch head being sized and shaped to be forced into the femoral condyle to thereby circumferentially cut off the bone plug.

13. The bone punch instrument according to claim 12, wherein the impaction handle comprises a cannulation extending longitudinally between the force receiving end and the punch head coupling end.

14. The bone punch instrument according to claim 12, wherein the punch head bore is delimited circumferentially by a wall, and wherein the punch head comprises at the cutting end of the punch head a compression surface formed by a punch head bore facing surface of the wall, the compression surface being circumferentially tapered toward a center of the punch head bore such that the compression surface is arranged to apply a compression force on the bone plug such that the compression force increases while the bone plug advances toward the impaction handle coupling end inside the punch head bore.

15. The bone punch instrument according to claim 12, wherein the punch head bore is delimited circumferentially by a wall divided longitudinally along the length of the punch head into a compression section at the cutting end, a coupling element section at the impaction handle coupling end, and a middle section between the compression section and the coupling element section, and wherein in the middle section the wall has a cross-sectional thickness between 0.3 mm and 1.0 mm.

16. The bone punch instrument according to claim 12, wherein first the first coupling element is a male thread and the second coupling element is a female thread or vice versa, and wherein the male thread is arranged to mate with the female thread to form a threaded connection.

17. The bone punch instrument according to claim 12, wherein the bone punch instrument further comprises a longitudinal, cannulated centralization element sized and shaped to be at least partially received in the punch head bore.

18. The bone punch instrument according to claim 17, wherein the centralization element is longitudinally divided into a first centralization element section characterized by a first centralization element outer diameter, and a second centralization element section characterized by a second centralization element outer diameter, which is smaller than the first centralization element diameter, and wherein the first centralization element section is sized and shaped to be slidingly and in a form-fit manner received within the punch head bore, and the second centralization element section is sized and shaped to be at least partially slidingly and in a form-fit manner received within an cannulation of the impaction handle.

19. The bone punch instrument according to claim 12, wherein the punch head bore is delimited circumferentially by a wall having openings and/or protrusions on an inner surface of the wall facing the punch head bore.

20. The bone punch instrument according to claim 12, wherein the bone punch instrument further comprises a punch head holder comprising a punch head holder bore sized and shaped to at least partially receive the punch head and the bone plug, the punch head holder bore comprising a stop element for preventing the punch head from moving beyond the stop element while allowing the bone plug to move beyond the stop element to enable removal of the bone plug out of the punch head.

21. A bone punch instrument kit for creating a bone tunnel in a femoral condyle, the bone punch instrument comprising
an impaction handle having a force receiving end and a punch head coupling end comprising a first coupling element;
a first and second hollow, elongated punch heads, the first and second punch heads having a cutting end and an impaction handle coupling end comprising a second coupling element configured to be removably coupled to the first coupling element such that a force exerted on the force receiving end of the impaction handle is configured to be transferred onto the punch head, the first and second punch heads comprising a punch head bore for receiving a bone plug of the femoral condyle, and a cutting edge arranged circumferentially around the bore at the cutting end of the punch head, the first and second punch heads being sized and shaped to be forced into the femoral condyle to thereby circumferentially cut off the bone plug, the first punch head having a first length and a first punch bore diameter, and the second punch head having a second length and a second punch bore diameter and wherein the first length is different from the second length and/or the first diameter is different from the second diameter.
